(19) United States
(12) Patent Application Publication
(10) Pub. No.: US 2024/0424044 A1
(43) Pub. Date: Dec. 26, 2024

(54) TRADITIONAL CHINESE MEDICINE COMPOUND PREPARATION WITH PURE HERBS FOR DETOXIFICATION AND FUKANG AND A PREPARATION METHOD THEREOF

(71) Applicant: Xuegan Du, Wuhan (CN)

(72) Inventor: Xuegan Du, Wuhan (CN)

(21) Appl. No.: 18/359,720

(22) Filed: Jul. 26, 2023

(30) Foreign Application Priority Data
Jun. 21, 2023 (CN) 2023107386168

(51) Int. Cl. A61K 36/258; A61K 9/48; A61K 36/232; A61K 36/233; A61K 36/237; A61K 36/284; A61K 36/285; A61K 36/46; A61K 36/481; A61K 36/484; A61K 36/65; A61K 36/66; A61K 36/71; A61K 36/714; A61K 36/718; A61K 36/725; A61K 36/752; A61K 36/756; A61K 36/815; A61K 36/835; A61K 36/8988; A61K 47/46; A61P 25/36; A61P 29/00

(52) U.S. Cl. CPC A61K 36/258; A61K 9/4833; A61K 36/232; A61K 36/233; A61K 36/237; A61K 36/284; A61K 36/285; A61K 36/46; A61K 36/481; A61K 36/484; A61K 36/65; A61K 36/66; A61K 36/71; A61K 36/714; A61K 36/718; A61K 36/725; A61K 36/752; A61K 36/756; A61K 36/815; A61K 36/835; A61K 36/8988; A61K 47/46; A61P 25/36; A61P 29/00; A61K 2236/15; A61K 2236/17

(57) ABSTRACT

A traditional Chinese medicine compound preparation with pure herbs for detoxification and Fukang, and a preparation method thereof, wherein the traditional Chinese medicine compound preparation for detoxification and Fukang includes the detoxification preparation, Fukang preparation, as well as dryness-removing and pain-relieving preparation; its preparation method includes Die Wan manufacturing process and capsule manufacturing process, the detoxification preparation and Fukang preparation adopt the Die Wan manufacturing process, the dryness-removing and pain-relieving preparation adopt the capsule manufacturing process; the invention is a traditional Chinese medicine compound preparation with pure herbs, which is a universal type for any single drug and mixed drugs, with short treatment period, quick effect, less cost, detoxification in one week, keeping people strong and healthy in another week, and no withdrawal syndrome, no drug dependence, no toxic side effects and good anti-relapse effect; it can greatly alleviate the pain of abrupt withdrawal during the detoxification process.

### 1. TECHNICAL FIELD

The invention relates to the technical field of drugs for detoxification, in particular to a traditional Chinese medicine compound preparation with pure herbs for detoxification and Fukang, and a preparation method thereof.

### 2. BACKGROUND ART

Modern medicine often uses “methadone” as a detoxification drug to curb withdrawal symptoms, and it is called “soft drug”. It will cause drug addicts to develop drug dependence during withdrawal treatment, and it will take time to gradually reduce the dose to get rid of addiction; other detoxification drug such as Anjunning and Naltrexone need to be taken for 3 months to several years, which take a long time and cost a lot of money, resulting in repeated detoxification failures and loss of confidence by drug addicts, resulting in difficulty in detoxification.

The China invention patent with the publication number CN 1077796C discloses a traditional Chinese medicine preparation for detoxification and Fukang, which consists of two parts: a detoxification preparation and a Fukang preparation. Although this traditional Chinese medicine preparation can effectively avoid drug addicts' dependence on drug detoxification drugs, improve and enhance immune function, and regulate the balance of yin and yang; it produces cliff-like withdrawal symptoms due to taking the detoxification drugs, it is very painful and uncomfortable, so that they stop taking the detoxification drugs midway, therefore, the detoxification process is interrupted, resulting in a failure of detoxification.

### 3. SUMMARY OF THE INVENTION

The technical problem to be solved by the invention is to provide a traditional Chinese medicine compound preparation with pure herbs for detoxification and Fukang, and a preparation method thereof, which has fast detoxification speed and can avoid cliff-like withdrawal pain.

To solve the above technical problems, the invention provides the following technical scheme:
A traditional Chinese medicine compound preparation with pure herbs for detoxification and Fukang includes the detoxification preparation, Fukang preparation, as well as dryness-removing and pain-relieving preparation;
The detoxification preparation is formula A, which comprises the following raw materials in parts by weight:
Radix panacis Quinquefolii 50-70 g, Rhizoma gastrodiae 30-35 g, Rhizoma cimicifugae 30-40 g, Radix aconiti lateralis praeparata 5-10 g, pericarpium papaveris 50-70 g, Radix et Rhizoma glycyrrhizae praeparata cum Melle 30-35 g, cortex phellodendri chinensis 40-45 g, Rhizoma coptidis 40-45 g, Radix aucklandiae 25-30 g, Radix astragali 30-40 g, Rhizoma atractylodis Macrocephalae 20-30 g, Radix angelicae sinensis 25-30 g, Radix bupleuri 20-35 g, pericarpium citri reticulatae 25-25 g, Lignum aquilariae resinatum 25-30 g;
The Fukang preparation is formula B, which comprises the following raw materials in parts by weight:
Radix panacis Quinquefolii 50-70 g, Rhizoma cimicifugae 30-40 g, Rhizoma atractylodis Macrocephalae 30-40 g, Radix bupleuri 15-20 g, Cortex eucommiae 30-40 g, roasted Rhizoma gastrodiae 10-20 g, fructus lycii 30-35 g, Radix angelicae sinensis 30-35 g, Lignum aquilariae resinatum 20-30 g, cortex phellodendri chinensis 40-45 g, Radix astragali praeparata cum Melle 30-40 g, Radix et Rhizoma glycyrrhizae praeparata cum Melle 30-35 g, Rhizoma coptidis 40-45 g, fried Semen ziziphi spinosae 25-30 g;
The dryness-removing and pain-relieving preparation is formula C, which comprises the following raw materials in parts by weight:
Rhizoma Corydalis 15-30 g, Radix paeoniae alba 10-15 g, Radix et Rhizoma Notopterygii 15-20 g.

Preferably, the detoxification preparation comprises the following raw materials in parts by weight:
Radix panacis Quinquefolii 70 g, Rhizoma gastrodiae 35 g, Rhizoma cimicifugae 30 g, Radix aconiti lateralis praeparata 10 g, pericarpium papaveris 50 g, Radix et Rhizoma glycyrrhizae praeparata cum Melle 35 g, cortex phellodendri chinensis 40 g, Rhizoma coptidis 45 g, Radix aucklandiae 30 g, Radix astragali 40 g, Rhizoma atractylodis Macrocephalae 30 g, Radix angelicae sinensis 30 g, Radix bupleuri 20 g, pericarpium citri reticulatae 25 g, Lignum aquilariae resinatum 30 g;
The Fukang preparation comprises the following raw materials in parts by weight:
Radix panacis Quinquefolii 70 g, Rhizoma cimicifugae 40 g, Rhizoma atractylodis Macrocephalae 40 g, Radix bupleuri 15 g, Cortex eucommiae 40 g, roasted Rhizoma gastrodiae 20 g, fructus lycii 35 g, Radix angelicae sinensis 35 g, Lignum Aquilariae Resinatum 30 g, cortex phellodendri chinensis 45 g, Radix astragali praeparata cum Melle 40 g, Radix et Rhizoma glycyrrhizae praeparata cum Melle 35 g, Rhizoma coptidis 40 g, fried Semen ziziphi spinosae 30 g;
The dryness-removing and pain-relieving preparation comprises the following raw materials in parts by weight:
Rhizoma Corydalis 30 g, Radix paeoniae alba 15 g, Radix et Rhizoma Notopterygii 20 g.

The invention also provides a preparation method of the above traditional Chinese medicine compound preparation for detoxification, its preparation method includes Die Wan manufacturing process and capsule manufacturing process, the detoxification preparation and Fukang preparation adopt the Die Wan manufacturing process, the dryness-removing and pain-relieving preparation adopt the capsule manufacturing process;

The Die Wan manufacturing process comprises the following steps:
Step 1. Inspection: carefully inspecting the formula, drug quality and quantity;
Step 2. Pre-treatment of Pulverization: sorting out the Chinese herbal medicines to be crushed, and putting them in a drying room or oven to dry according to different requirements; drying the medicines at 80° C. for 48 hours, during which the medicines were turned 2-3 times to prevent uneven drying and paste;
Step 3. Pulverization: cutting the dried medicines into the same size and mixing them evenly for pulverization; then putting them into a pulverizer to pulverize and sift; repeatedly pulverizing and sifting until all the medicine powder reaches a fineness of 80-100 mesh;
Step 4. Die Wan: according to the weight of the medicines and the size of the ready-made medicine, preparing a certain amount of medicine, the method is as follow: putting a small amount of medicine powder into the sugar-coated pan, spraying water on the powder with a sprayer, starting the pan, using the rotation of the pan and manual kneading to make the medicine powder evenly moisten, stopping the water and adding powder, so that the medicine powder becomes fine particles, sifting repeatedly in this way to shape the medicinal pills; then washing and drying the pot, putting it into the medicinal base, dissolving the pot washing water and the remaining materials from the preparation of medicinal base together into a small water paste, for honey pills, adding honey to the honey pill and dissolving it for later use; using slurry or clean water to moisten the medicinal base, then adding powder and rotating continuously to make the medicine powder evenly adhere to the medicinal base; in this way, adding water, adding powder, sifting evenly, and repeating these operations in sequence until the powder is used up, the size of the pills is uniform, and the surface is smooth and appreciable;
Step 5. Drying and Shaping: placing the stacked pills into a drying room or oven for drying, the pills must be dried in an environment at 80° C. for 48 hours, after thorough drying, sifting and shaping them;
Step 6. Moistening and Polishing with water: after the pills are shaped and dried, their surface colors may appear different, therefore, the pills need to be moistened with distilled water, this process involves placing the pills into a pot and adding an appropriate amount of water, allowing all pill surfaces to come into contact with the water, after that, removing the pills from the pot and drying them again in an environment at 80° C. for 12 hours, finally, they can be bottle-packaged.

The capsule manufacturing process comprises the following steps:
Step 1. Inspection: carefully inspecting the formula, drug quality and quantity;
Step 2. Pre-treatment of Pulverization: sorting out the Chinese herbal medicines to be crushed, and putting them in a drying room or oven to dry according to different requirements; drying the medicines at 80° C. for 48 hours, during which the medicines were turned 2-3 times to prevent uneven drying and paste;
Step 3. Pulverization: cutting the dried medicines into the same size and mixing them evenly for pulverization; then putting them into a pulverizer to pulverize and sift; repeatedly pulverizing and sifting until all the medicine powder reaches a fineness of 80-100 mesh;
Step 4. Using the capsule filling machine to inject the medicine powder into the “0” capsule;
Step 5. Packaging them in medicine bottles, sealing and saving.

Compared with the prior art, the invention has the following advantages: the invention is a traditional Chinese medicine compound preparation with pure herbs, which is a universal type for any single drug and mixed drugs, with short treatment period, quick effect, less cost, detoxification in one week, keeping people strong and healthy, enhancing immune function in another week, and no withdrawal syndrome, no drug dependence, no toxic side effects and good anti-relapse effect; it can greatly alleviate the pain of abrupt withdrawal during the detoxification process, and it can realize easy and painless detoxification and Fukang.

### 4. BRIEF DESCRIPTION OF ACCOMPANY DRAWINGS

FIG. 1 is an analysis diagram of the theory of drug addiction.

FIG. 2 is an analysis diagram of the medical theory of treating drug addiction.

FIG. 3 is an illustration of the common reasons why other types of detoxification drugs cost a lot of money, take a long time, and are difficult to detoxify.

### 5. SPECIFIC EMBODIMENT OF THE INVENTION

A traditional Chinese medicine compound preparation with pure herbs for detoxification and Fukang, and a preparation method thereof of the invention will be further described below in combination with accompanying drawings.

### Principle of the Invention:

The invention is derived from the theoretical basis of traditional Chinese medicine, and is explained in a typical way of taking drugs. There are many ways to ingest drugs, such as inhaling, eating, and injecting, but patients who become drug addicts all have basically the same physical and mental symptoms. The treatment principle of the Chinese medicine compound preparation of the invention is both interlinked and universal to all patients after forming drug addiction. Combined with the illustration of taking drugs as a typical example and “inhaling and eating” as a representative, the theoretical basis of traditional Chinese medicine of the invention is explained as follow.

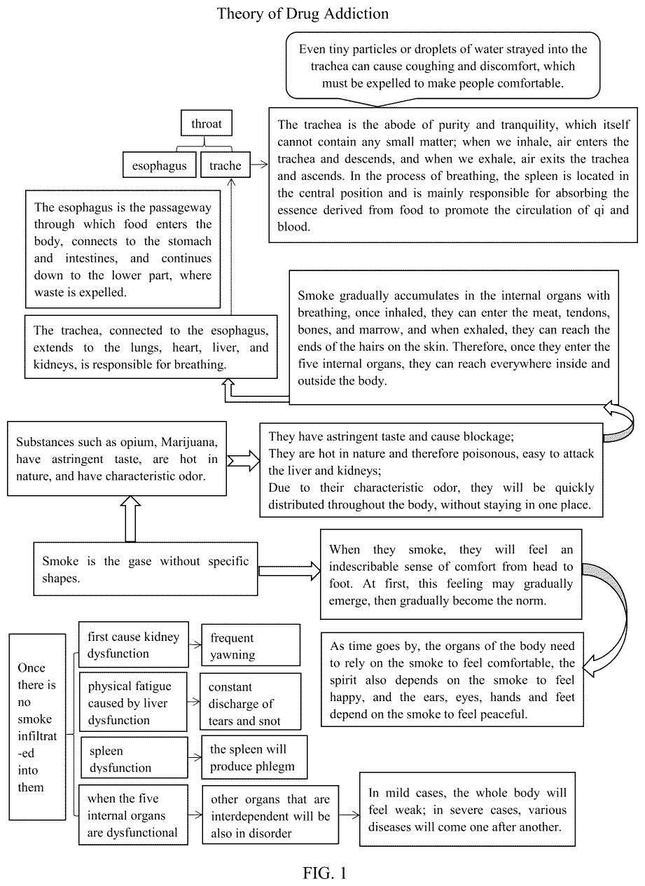

Combined with FIG. 1: Theory of Drug Addiction

The human throat can be divided into two parts: the esophagus and the trachea. The esophagus is the passageway through which food enters the body, connects to the stomach and intestines, and continues down to the lower part, where waste is expelled. The trachea, connected to the esophagus, is responsible for breathing, and extends to the lungs, heart, liver, and kidneys. When we inhale, air enters the trachea and descends, and when we exhale, air exits the trachea and ascends. In the process of breathing, the spleen is located in the central position and is mainly responsible for absorbing the essence derived from food to promote the circulation of qi and blood. However, if harmful substances such as smoke is inhaled, it will enter the body through the trachea, and accumulate in the five internal organs to form dirt with the reciprocating movement of breathing. Different from the stomach and esophagus, which receive food and drink, the trachea is the abode of purity and tranquility, which itself cannot contain any small matter. Even tiny particles or droplets of water strayed into the trachea can cause coughing and discomfort, which must be expelled to make people comfortable. Smoke is the gase without specific shapes, which gradually accumulate in the internal organs with breathing, while substances such as opium and morphine have astringent taste and cause blockage. They are hot in nature and therefore poisonous. They are easy to attack the liver and kidneys. Due to their characteristic odor, these substances will be quickly distributed throughout the body, without staying in one place. Once inhaled, they can enter the meat, tendons, bones, and marrow, and when exhaled, they can reach the ends of the hairs on the skin. Therefore, once they enter the five internal organs, they can reach everywhere inside and outside the body. Observing those who are addicted to smoking, when they smoke, they will feel an indescribable sense of comfort from head to foot. At first, this feeling may gradually emerge, then gradually become the norm. As time goes by, the organs of the body need to rely on the smoke to feel comfortable, the spirit also depends on the smoke to feel happy, and the ears, eyes, hands and feet depend on the smoke to feel peaceful. Once there is no smoke infiltrated into them, it will first cause kidney dysfunction, and kidney dysfunction can lead to frequent yawning. Secondly, it will lead to physical fatigue caused by liver dysfunction, which will cause constant discharge of tears and snot. Moreover, the spleen will produce phlegm, which further lead to corresponding symptoms of spleen dysfunction. When the five internal organs are dysfunctional, other organs that are interdependent will be also in disorder, and various physical problems will occur at the same time, in mild cases, the whole body will feel weak; in severe cases, various diseases will come one after another, and almost no disease will not attack. That's what's called a drug addict. Now that we know the cause of addiction, we should know the method of medication, which is to regulate the spleen and stomach and diet. For those with mild addictions and those with strong physiques, only effective prescription can help them quit their addiction.

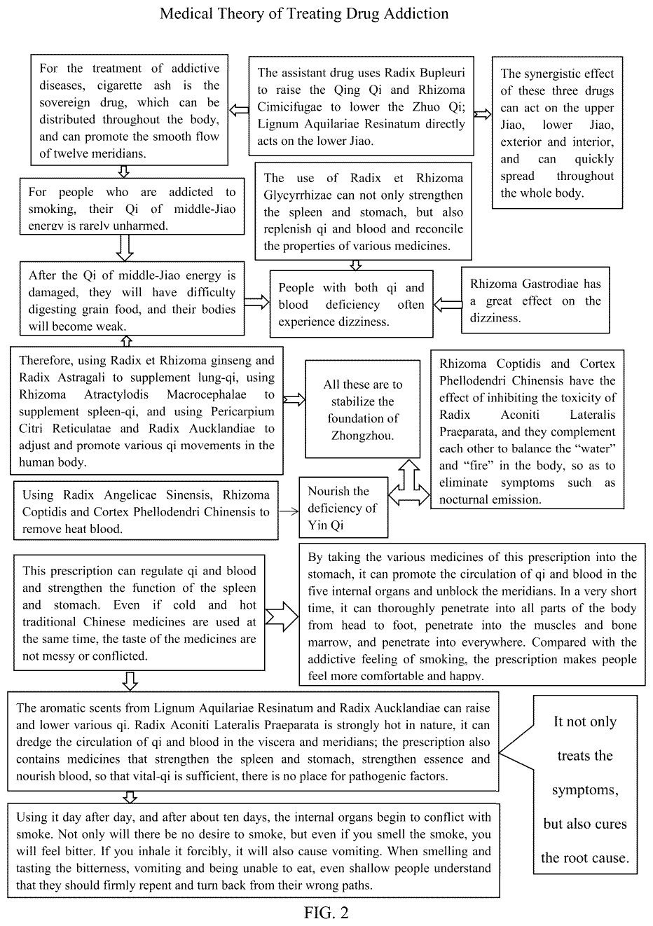

Combined with FIG. 2: Medical Theory of Treating Drug Addiction

For the treatment of addictive diseases, cigarette ash is the sovereign drug, and Radix aconiti lateralis praeparata is used as an auxiliary, which can be distributed throughout the body, and can promote the smooth flow of twelve meridians; the assistant drug uses Radix bupleuri to raise the Qing Qi and Rhizoma cimicifugae to lower the Zhuo Qi; Lignum aquilariae resinatum directly acts on the lower Jiao, and the synergistic effect of these drugs can act on the upper Jiao, lower Jiao, exterior and interior, and can quickly spread throughout the whole body. For people who are addicted to smoking, their Qi of middle-Jiao energy is rarely unharmed. After the Qi of middle-Jiao energy is damaged, they will have difficulty digesting grain food, and their bodies will become weak. Therefore, using Radix et Rhizoma ginseng and Radix astragali to supplement lung-qi, using Rhizoma atractylodis Macrocephalae to supplement spleen-qi, and using pericarpium citri reticulatae and Radix aucklandiae to adjust and promote various qi movements in the human body, all these are to stabilize the foundation of Zhongzhou. Once the foundation is firm, using Radix angelicae sinensis, Rhizoma coptidis and cortex phellodendri chinensis to remove heat blood and nourish the deficiency of Yin Qi. Rhizoma coptidis and cortex phellodendri chinensis have the effect of inhibiting the toxicity of Radix aconiti lateralis praeparata, and they complement each other to balance the “water” and “fire” in the body. People with both qi and blood deficiency often experience dizziness, Rhizoma gastrodiae has a great effect on the dizziness, so it is necessary to add Rhizoma gastrodiae. The use of Radix et Rhizoma glycyrrhizae can not only strengthen the spleen and stomach, but also replenish qi and blood and reconcile the properties of various medicines. This prescription can regulate qi and blood and strengthen the function of the spleen and stomach. Even if cold and hot traditional Chinese medicines are used at the same time, the taste of the medicines are not messy or conflicted. This is the principle of prescription. This prescription has a wonderful effect on smoking cessation, and the effect is too wonderful to express in words. By taking the various medicines of this prescription into the stomach, it can promote the circulation of qi and blood in the five internal organs and unblock the meridians. In a very short time, it can thoroughly penetrate into all parts of the body from head to foot, penetrate into the muscles and bone marrow, and penetrate into everywhere. Compared with the addictive feeling of smoking, the prescription makes people feel more comfortable and happy. After using the prescription, various diseases will not attack, and the addiction to smoking will not appear again. The prescription contains aromatic scents from Lignum aquilariae resinatum and Radix aucklandiae, and their aromatic scents can raise and lower various qi. At the same time, because Radix aconiti lateralis praeparata is strongly hot in nature, it can dredge the circulation of qi and blood in the viscera and meridians. Using it day after day, and after about ten days, the internal organs begin to conflict with smoke. Not only will there be no desire to smoke, but even if you smell the smoke, you will feel bitter. If you inhale it forcibly, it will also cause vomiting. When smelling and tasting the bitterness, vomiting and being unable to eat, even shallow people understand that they should firmly repent and turn back from their wrong paths. In addition, the prescription also contains medicines that strengthen the spleen and stomach, strengthen essence and nourish blood. Using “DetoxKang” A20 g+C of the invention every day to reduce addiction. After 6 days, changing to take “DetoxKang” B20 g+C to supplement vital-qi. When vital-qi is sufficient, there is no place for pathogenic factors. Whether it is a serious addiction or a debilitating physical condition, when treated in this prescription, it is always possible to achieve a pure and clear understanding, making the process of overcoming them relatively easy. Although some people have said that even if some people take effective drugs, they can stop smoking but don't stop, they can quit but don't want to quit, lie on the pillow under the light, and have a strong attachment to opium, then drug addiction may be easily cured, but psychological dependence is difficult to heal, just like willing to become an ulcer attached to the bone, which can only be relieved after death. No matter how miraculous the elixir is, it cannot change this situation. May all those who have aspirations, once they have cleansed their intestines and stomach, regard drugs as an irreconcilable enemy, so that they will not let down the choice of “DetoxKang” (a traditional Chinese medicine compound preparation with pure herbs).

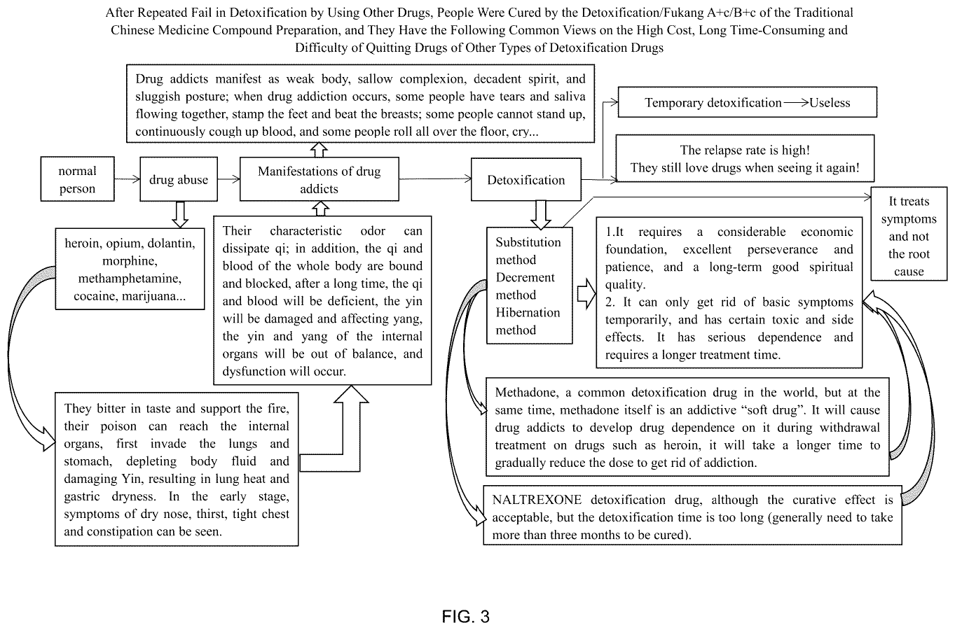

Combined with FIG. 3, after repeated fail in detoxification, people tried the “DetoxKang” of the invention and were cured within 12 days, they have the following common views on the high cost, long time-consuming and difficulty of quitting drugs of other types of detoxification drugs:

“DetoxKang”, a traditional Chinese medicine compound preparation with pure herbs, ignites hope in drug addicts. The invention has the principle of sovereign, minister, assistant, and courier, which makes traditional Chinese medicines well arranged, and it has unique curative effect, the treatment period is only 12 days. It can not only quit the addiction, strengthen the body resistance, and recover, but there is no need to reduce the dose of detoxification drugs, and the curative effect is very satisfactory.

Every drug addict who agrees to take “DetoxKang” has experienced multiple unsuccessful attempts at drug rehabilitation, leading to complete disappointment with all existing drug rehabilitation methods. They tried “DetoxKang” as a last resort to save a hopeless situation. Their results are generally that miracles happened. Sure enough, they quit the addiction, strengthened the body resistance, and recovered within 12 days. They finally raised the sails of life with renewed confidence.

The traditional Chinese medicine compound preparation of “DetoxKang” of the invention is unique, miraculously effective, safe and fixed. The basic pharmacology and the explanation of this prescription are as follows.

The basic pharmacology of “DetoxKang”:

Clearing away heat, detoxifying and expelling toxins, aromatic and refreshing, removing turbidity, it has a bidirectional regulatory effect on vegetative nerve function, and effectively prevent the occurrence of withdrawal symptoms. Strengthening the body resistance and dispelling pathogen factors, nourishing blood and calming the nerves, improving and enhancing the immune function of drug addicts, quickly adjusting the balance of yin and yang in patients, nourishing qi and blood, so as to eliminate the root cause of addiction. The special effect of “DetoxKang”: after being treated by “DetoxKang” (a traditional Chinese medicine compound preparation with pure herbs), drug addicts feel disgusted by drugs and no longer have the idea of taking drugs, which promotes rapid recovery and achieves the object of drug rehabilitation.

The specific explanation of this prescription: when taking drugs, it can relax the whole body, make people feel intoxicated and ecstatic, which is very beneficial. If you smoke three times in a row to form a drug addiction, and if you don't continue to smoke, your whole body will convulse, your muscles and vessels will spasm and contract, your tears and nasal mucus will flow together, you will have unbearable abdominal pain, and opisthotonos, it will be extremely painful. Drugs are extremely harmful hot substances, most capable of burning the Yang Qi of the human body, increasing the fire and depleting Qi, causing damage to the spleen and lungs. Long-term damage to the spleen can lead to muscle atrophy, which makes the body's muscles thin, and symptoms such as shortness of breath and mental fatigue appear, therefore, using Radix et Rhizoma ginseng and Radix astragali to nourish Qi, and Rhizoma atractylodis Macrocephalae to nourish the spleen; when the Qi is weak, the blood will become deficient, therefore, using Radix angelicae sinensis and Radix paeoniae alba to generate the blood; the heat-toxin attacks the heart and kidneys, therefore, using Rhizoma coptidis to relieve the heart fire, using cortex phellodendri chinensis to relieve the kidney fire, using Rhizoma gastrodiae and Rhizoma cimicifugae to raise the Qing Qi and using Lignum aquilariae resinatum to lower the Zhuo Qi, Radix bupleuri acts on the liver and relieves spasms and cramps, pericarpium citri reticulatae and Radix aucklandiae have the effect of regulating qi, the smooth flow of qi can make the internal organs unobstructed, Rhizoma Corydalis can relieve pain and emergency, Radix et Rhizoma glycyrrhizae can detoxify and strengthen the spleen and stomach, combined with Radix aconiti Lateralis praeparata, this is for treating false-heat syndrome with this hot natured drug, using Radix et Rhizoma Notopterygii to warm the blood and body fluids running in the sun meridian. However, all of these can be rejected by the meridians and viscera with deep drug addiction and are out of place. Therefore, a large dose of pericarpium papaveris is used to introduce the above-mentioned drugs into it, so that the addict can be half-drunk and half-awake, and the medicinal efficacy will act on the place where the drug addiction is lying, so the drug addiction can be withdrawn. Since people cannot stay in the half-drunk and half-awake state for a long time, replacing pericarpium papaveris with the Fuzheng preparation one by one, and finally the drug addiction is completely withdrawn. The whole preparation has the synergistic effects of strengthening the synergistic and purging toxins, soothing the liver and regulating qi, so there is no worry about relapse.

The specific implementation process of a traditional Chinese medicine compound preparation with pure herbs for detoxification and Fukang, and a preparation method thereof of the invention is as follow:

Formula A-a detoxification preparation (the preparation for withdrawing addiction and detoxification): after processing the following 15 Chinese herbal medicines, grinding them into powder, mixing and shaping.

The specific pharmaceutical steps are as follows:
Step 1. Inspection: carefully inspecting the formula, drug quality and quantity; selecting Radix panacis Quinquefolii 70 g, Rhizoma gastrodiae 35 g, Rhizoma cimicifugae 30 g, Radix aconiti lateralis praeparata 10 g, pericarpium papaveris 50 g, Radix et Rhizoma glycyrrhizae praeparata cum Melle 35 g, cortex phellodendri chinensis 40 g, Rhizoma coptidis 45 g, Radix aucklandiae 30 g, Radix astragali 40 g, Rhizoma atractylodis Macrocephalae 30 g, Radix angelicae sinensis 30 g, Radix bupleuri 20 g, pericarpium citri reticulatae 25 g, Lignum aquilariae resinatum 30 g;
Step 2. Pre-treatment of Pulverization: sorting out the Chinese herbal medicines selected in step 1, and putting them in a drying room or oven to dry according to different requirements. Drying the medicines at 80° C. for 48 hours, during which the medicines were turned 2-3 times to prevent uneven drying and paste;
Step 3. Pulverization: cutting the dried medicines into the same size and mixing them evenly for pulverization. Then putting them into a pulverizer to pulverize and sift. Generally, pills require a fineness of 80-100 mesh. Repeatedly pulverizing and sifting until all the medicine powder reaches a fineness of the mesh.
Step 4. Die Wan: according to the weight of the medicines and the size of the ready-made medicine, preparing a certain amount of medicine, the method is as follow: putting a small amount of medicine powder into the sugar-coated pan, spraying water on the powder with a sprayer, starting the pan, using the rotation of the pan and manual kneading to make the medicine powder evenly moisten, stopping the water and adding powder, so that the medicine powder becomes fine particles, sifting repeatedly in this way to shape the medicinal pills. Then washing and drying the pot, putting it into the medicinal base (dissolving the pot washing water and the remaining materials from the preparation of medicinal base together into a small water paste, for honey pills, adding honey to the honey pill and dissolving it for later use). Using slurry or clean water to moisten the medicinal base, then adding powder and rotating continuously to make the medicine powder evenly adhere to the medicinal base; in this way, adding water, adding powder, sifting evenly, and repeating these operations in sequence until the powder is used up, the size of the pills is uniform, and the surface is smooth and appreciable.
Step 5. Drying and Shaping: placing the stacked pills into a drying room or oven for drying, the pills must be dried in an environment at 80° C. for 48 hours, after thorough drying, sifting and shaping them;
Step 6. Moistening and Polishing with water: due to the complicated production process, after the pills are shaped and dried, their surface colors may appear different, therefore, the pills need to be moistened with clean water, the quality of water used should be distilled water. This process involves placing the pills into a pot, aftering boiling, stirring while adding water (adding an appropriate amount of water, allowing all pill surfaces to come into contact with the water), after that, removing the pills from the pot and drying them again in an environment at 80° C. for 12 hours. This has achieved the objects of sterilizing and shaping. In this way, they can be bottle-packaged.

Formula B-a Fukang preparation (the preparation for Fuzheng: strengthening the body resistance and Ningshen: calming the mind), after processing the following 14 Chinese herbal medicines, grinding them into powder, mixing and shaping.

Its specific production steps are the same as that of Formula A-a detoxification preparation, only the selection of medicinal materials is different. This Fuzheng and Ningshen preparation selects Radix panacis Quinquefolii 70 g, Rhizoma cimicifugae 40 g, Rhizoma atractylodis Macrocephalae 40 g, Radix bupleuri 15 g, Cortex eucommiae 40 g, roasted Rhizoma gastrodiae 20 g, fructus lycii 35 g, Radix angelicae sinensis 35 g, Lignum aquilariae resinatum 30 g, cortex phellodendri chinensis 45 g, Radix astragali praeparata cum Melle 40 g, Radix et Rhizoma glycyrrhizae praeparata cum Melle 35 g, Rhizoma coptidis 40 g, fried Semen ziziphi spinosae 30 g;

Formula C-a dryness-removing and pain-relieving preparation, after processing the following 3 Chinese herbal medicines, grinding them into powder, mixing and shaping.

The specific pharmaceutical steps are as follows:
Step 1. Inspection: carefully inspecting the formula, drug quality and quantity; selecting Rhizoma Corydalis 30 g, Radix paeoniae alba 15 g, Radix et Rhizoma Notopterygii 20 g.
Step 2. Pre-treatment of Pulverization: sorting out the Chinese herbal medicines selected in step 1, and putting them in a drying room or oven to dry according to different requirement. Drying the medicines at 80° C. for 48 hours, during which the medicines were turned 2-3 times to prevent uneven drying and paste.
Step 3. Pulverization: cutting the dried medicines into the same size and mixing them evenly for pulverization; then putting them into a pulverizer to pulverize and sift. Generally, pills require a fineness of 80-100 mesh. Repeatedly pulverizing and sifting until all the medicine powder reaches a fineness of the mesh.
Step 4. Using the capsule filling machine to inject the medicine powder into the “0” capsule.
Step 5. Packaging them in 80 ml medicine bottles (90 capsules per bottle), sealing and saving with a shelf life of three years.

### Usage and Dosage:

Taking DetoxKang-A for half an hour before meals, four times a day (once every six hours), 20 g each time, taking with warm water, and taking DetoxKang-C for half an hour after meals, three times a day, three capsules each time, after taking it for six days, taking DetoxKang-B for half an hour before meals, four times a day (once every six hours), 20 g each time, taking with warm water, and continuing to take DetoxKang-C for half an hour after meals, three times a day, three capsules each time.

Typical Cases and Treatment Process with Human Use Experience

In the human use experience of “DetoxKang”, the conclusive summary of the statistical data of the rescued persons published in the newspaper and the drug addicts in the drug rehabilitation center of the Public Security Bureau, as well as the investigation of the relapse rate are as follows:

### 1. Rescued Person Published in Newspapers

Wuhan Evening News, October 24th to Nov. 8, 2003: The Call for Social Salvation in “Saving A Drug Addicted Girl”, and “Xiaohui's Drug Rehabilitation Diary”, which was followed up and reported by reporters for 12 days, was serialized. The facts and statistics recorded are briefly described as follows:
\textcircled{1} Xiaohui failed to detoxify in the drug rehabilitation center 4 times in the early stage, which proved that all kinds of traditional Chinese medicine and western medicine were ineffective for her detoxification.
\textcircled{2} After taking “DetoxKang”, the unique amd miraculous effect, safety, and clinical benefits in human experience of “DetoxKang” have been verified. The effect of 12-day detoxification is withdrawing addiction and detoxification with ease and no pain in the first 6 days; consolidating the curative effect and quickly recovering physical fitness in next 6 days. The time is short, the effect is quick, and the traditional Chinese medicine compound preparation with pure herbs has no toxic side effects and drug dependence reactions. On October 26th, the first day, she took A+C. On the 27th, Xiaohui's Drug Rehabilitation Diary said: “The second day of detoxification . . . unlike the previous detoxification, I used to feel uncomfortable all over the body, like an ant biting my bones, I felt itchy and painful; but now I just feel painful but not itchy.” On October 30th, “Today is the fifth day of detoxification, the most difficult first three days of detoxification have passed, and I feel much better.” On November 1st, the 7th day, I switched to prescription B+C. “undergoing recovery treatment . . . starting physical therapy of Fuzheng (strengthening the body resistance), calming the heart, and calming the nerves.” On November 3rd, “Today is the third day of Fuzheng, my family members say that my complexion is much better than the previous few days. I also feel very good and calm. Traditional Chinese medicine focuses on conditioning, and the effect is obvious these days.” On November 7th, “Today is the last day of this detoxification course. After these 12 days of treatment, I feel much better in spirit, in good physical condition, and I have gained weight. . . . When I think of the upcoming new life, my heart is filled with anticipation.”
\textcircled{3} The conclusion of the treatment statistical data in the public gaze: within 3 days, a physical body and mental body that has been immersed in drugs for a long time can be detoxified preliminarily; within 7 days, the transition from the rapid addiction withdraw and detoxification to the Fuzheng rehabilitation course can be basically completed; within 12 days, the drug rehabilitation and Fuzheng rehabilitation can be safely and effectively completed. It has short time, quick effect, no toxic side effects or drug dependence. You can turn around gloriously and bid farewell to drugs, toast to the past of drug use!!

### 2. Summary of Comprehensive Statistical Data on Curative Effect of Cases in Drug Rehabilitation Centers of Public Security Bureaus Across Five Provinces

From Aug. 27, 2002 to Nov. 9, 2007, Fujian Province (4 persons), Guangdong Province (2 persons), Henan Province (2 persons), Hubei Province (2 persons), Hainan Province (80 persons), a total of five provincial Public Security Bureau drug rehabilitation centers about the curative effect of “DetoxKang” traditional Chinese medicine preparation. A total of 90 people. The commonalities are as follows:
\textcircled{1} Generally, they are drug users who have tried many kinds of drug detoxification drugs, but have not succeeded.
\textcircled{2}) On the 3rd day after taking “DetoxKang” A+C, the symptoms of drug addiction were significantly relieved; after 4-6 days, the appetite increased, there was a desire to exercise, and the symptoms of drug addiction gradually disappeared; on the 7th day, switching to B+C, the mental state improved significantly, the eyes were bright, the intake and excretion were normal, and the behavior was normal, and the drug addiction did not occur again, and the urine and blood tests were all negative. The transition from the rapid addiction withdraw and detoxification to the Fuzheng rehabilitation course can be basically completed. During the 12 days, all the drug users achieved fast addiction withdraw and detoxification in the first 6 days, relaxed and painless; in the next 6 days, they strengthened the body resistance and calmed the mind, consolidated the curative effect, quickly recovered physical fitness, adjusted physiological functions, and improved immunity. Because it is a traditional Chinese medicine compound preparation with pure herbs, it has no damage to the internal organs of the human body and the nervous system of the brain, and it not only treats the symptoms, but also cures the root cause. No toxic side effects and drug dependence reactions were found after drug withdrawal. The unique miraculous curative effect of “DetoxKang” A+C/B+C traditional Chinese medicine compound preparation has been verified. In particular, it is worth pointing out that the Drug Rehabilitation Center of the Public Security Bureau of Chengmai County, Hainan Province. It can be seen from the materials on Aug. 27, 2006 that they selected He, Liu, and Cai who had taken drugs for 5-6 years and failed to detoxify with “Methadone” and “Anjunning” many times, as the first trial users of “DetoxKang”. After all three of them completed drug rehabilitation within 12 days, the drug rehabilitation center issued a certificate saying that the drug is extremely safe; it can truly achieve home drug rehabilitation.” Thereafter, in November, December, and January of the following year, the drug rehabilitation center completed two batches of 10-20 people's “graduation farewell parties” every month, because they successfully detoxified within 12 days. Together with the 3 people who tried it in the early stage, a total of 8 batches, a total of 80 people, completed the detoxification within 12 days.

Some cases are attached below:

### Case 1

Mr. Xiao, male, 40 years old, Han nationality, a compulsory drug addict in Zhangzhou City, Fujian Province, who lives in Meidao Town, Changle City, Fujian Province. He said that he had taken heroin for nearly ten years, about 1 g a day, and had been detoxified many times but failed to completely quit. On Aug. 19, 2002, he was sent to our institute for compulsory detoxification by the anti-drug brigade of Zhangzhou Public Security Bureau, Fujian.

On the morning of the 21st, his drug addiction broke out, tears and saliva flowed together, stamping the feet and beating the breast, and the whole body was sore. He took “DetoxKang A and C” orally. Four hours later, the body aches were relieved, there was no stool, the urination was normal, and he didn't want to eat.

On the 22nd, he continued to take “DetoxKang A and C”, the body still felt unwell, with runny nose, cough, etc, but the tearing phenomenon was alleviated.

On the 23rd, the symptoms mentioned above were obviously improved. He had one loose bowel movement, two meals of liquid food, the amount was 1/2 of the normal person's, and his whole body was weak.

On the 24th, he continued to take “DetoxKang A and C”. He wanted to eat at noon, in the afternoon, his drug addiction symptoms were mild, and the urination was normal.

From the 25th to the 26th, he continued to take it, and felt good about myself, with a little soreness all over the body, a normal diet, a sense of hunger, a desire to exercise, normal bowel movements and normal urination.

On the 27th, he changed to taking “DetoxKang B and C”. By the 30th, his spirit had improved significantly, his feet were strong, his speech was smooth, his body ache disappeared, and his diet, bowel movements and urination were normal.

From September 1st to 2nd, it was consolidated for two days, and the drug addiction did not show up again after three days. Through observation, the curative effect of “DetoxKang A, B and C” taken orally by the drug addict is remarkable. The first 6 days is the detoxification and withdrawal stage, and the drug addiction is reduced day by day, and his body gradually becomes better; the next 6 days is the recovery stage.

This product is a traditional Chinese medicine compound preparation with pure herbs, has no side effects after trial, and has remarkable curative effect, short treatment cycle, quick effect, rapid recovery, and no dependence on detoxification drugs.

### Case 2

Mr. He, male, Han nationality, 35 years old, from the Jinjiang Town, Chengmai County, Hainan Province, a compulsory detoxification personnel in Chengmai County Public Security Bureau's Drug Rehabilitation Center, he said had taken “heroin” for 6 years, about 1 g each time, he underwent compulsory detoxification and voluntary detoxification, but he failed many times. On Jun. 17, 2006, he was sent to our institute for compulsory detoxification by the anti-drug brigade of Chengmai County Public Security Bureau. When the drug addict first entered the drug rehabilitation center, he was irritable and his whole body was sore. Sometimes he felt that there were thousands of ants devouring him all over his body, and he moaned in pain. He was really uncomfortable. On the afternoon of August 15th, he took DetoxKang “A and C” orally. After 4-5 hours, his body felt a little hot, there was no stool, and the blood pressure and pulse were normal.

On August 16th, he took DetoxKang “A and C” orally, and he felt that his soreness was relieved, but the whole body was still weak. He started to have diarrhea, and the blood pressure and pulse were normal.

On August 17th, he continued to take DetoxKang “A and C”, and the curative effect was obviously improved, the symptoms of addiction were significantly relieved, the urine was normal, and the stool was loose 2-3 times.

From August 18th to 20th, he continued to take DetoxKang “A and C”. He ate normally, his body aches and drug addiction symptoms disappeared, and he felt good about himself.

From August 21st to 25th, the drug addict changed to taking DetoxKang “B and C”, he felt in good spirits, behaved like a normal person, ate normally, urinated and defecated 1-2 times, moved vigorously, and had normal blood pressure and pulse.

After taking “B and C” for 2 consecutive days, the drug addict wanted to exercise, felt energetic, drug addiction disappeared, blood pressure and pulse were normal.

After nearly two weeks of treatment and observation, the drug addict took “DetoxKang A, B, C” orally and the curative effect was remarkable, especially in the first week, the drug was detoxified, the addiction was removed, and it was harmless to the body. The next week was to regulate physiological functions and consolidate the curative effect, the body has fully recovered.

This medicine is a traditional Chinese medicine preparation with pure herbs, has a short course of treatment, quick treatment, quick effect, and no toxic and side effects on the body.

### Case 3

Mr. Liu, male, Han nationality, 25 years old, is from Jinjiang Town, Chengmai County, Hainan Province, a drug addict in the drug rehabilitation center of Chengmai County Public Security Bureau, he said he had a history of five years of drug use, 1 gram to 1.5 gram each time. During the period of drug use, he developed from “chasing the dragon” to the intravenous drug user. He had detoxified many times in our institute, and three times in the Hospital of Hainan Frontier Corps; in addition, he also bought “Methadone” and “Anjunning” at home to detoxify by himself, but none were successful. On Feb. 23, 2006, he was sent to our institute for detoxification by the County Public Security Bureau. When he first entered the the drug rehabilitation center, he was thin and dizzy, and after the onset of drug addiction, he felt fatigued, yawned, sweated, runny nose, and severe vomiting, especially the uncomfortable feeling like “worms crawling” in his body. On the afternoon of August 15th, he took DetoxKang “A and C”. Six hours later, the body was a little hot, and the lower abdomen was wriggling. on August 16th, after taking the medicine, the symptoms of drug addiction were alleviated, and the blood pressure and pulse were normal. From August 17th to August 20th, he continued to take “A and C”. He felt strong, wanted to exercise, the blood pressure and pulse were normal, his diet increased greatly, and he defecated 2-3 times a day. On August 21st, he took DetoxKang “B and C” for 4 consecutive days. The patient felt energetic, had a better diet, normal bowel movements and normal urination, and strong speech. After taking DetoxKang “B and C” for 2 consecutive days, the patient felt full of energy, behaved like a normal person, and his face was full of radiance.

After 12 days of treatment and observation, the drug addict took “DetoxKang A, B, C” orally and the curative effect was remarkable, especially in the first 6 days before taking the medicine, the drug was detoxified, the addiction was removed, his body was more relaxed, it was harmless to the body. In the next 6 week after taking the medicine, the physical fitness was strengthened, the energy was better, the mind was clear, and all returned to health. This “DetoxKang” is a traditional Chinese medicine preparation. Compared with other drug rehabilitation drugs, this drug has a short treatment time, quick effect, and no toxic and side effects on the body.

### Case 4

Mr. Cai, male, Han nationality, 35 years old, is from Jinjiang Town, Chengmai County, Hainan Province, a drug addict in the drug rehabilitation center of Chengmai County Public Security Bureau, he said he had a history of six years from “chasing the dragon” to the intravenous drug user, dark circles on his face, 1 gram to 1.5 gram everyday. He has undergone multiple drug rehabilitation attempts, has also taken the drugs “Methadone” and “Anjunning”, but they had no effect. On May 15, 2006, he was sent to our institute for detoxification by the County Public Security Bureau. When he first entered the the drug rehabilitation center, he was in poor health, his whole body was sore and limp, his face was pale, his body was thin, his expression was indifferent, black around his eyes, and he had poor appetite.

On the afternoon of August 15th, 5 hours after taking DetoxKang (A and C), his body became hot, and there was no stool, urinating twice, and the blood pressure was normal and regular. From August 16th to August 20th, he took DetoxKang “A and C” orally for 5 consecutive days. The patient felt energetic, had good appetite, slept well, the blackness around the eyes gradually faded, he defecated 2-3 times a day, urinated normally, the blood pressure was normal, his pulse was normal, and he had quit drug addiction.

Through observation, the curative effect of “DetoxKang A, B and C” taken orally by the drug addict is remarkable. The first 6 days is the detoxification and withdrawal stage; in the next 6 days is the Fuzheng and Ningshen stage, which can regulate physiological functions, strengthen the body, so as to achieve the object of reducing the relapse.

The medicine is extremely safe, and can truly realize family drug rehabilitation.

### 3. Survey on Relapse Rate

From November 6th to 10th, 2008, a follow-up survey was conducted on the relapse rate and status of drug addicts who took “DetoxKang” in the drug rehabilitation center of Chengmai County Public Security Bureau from 2006 to 2007. The summary of the statistical data shows that among the 27 respondents, only 3 patients relapsed after half a year. The non-relapse rate reached 81.48%.

The interpretation of TCM theory about the universal “detoxification/Fukang A+C/B+C traditional Chinese medicine compound preparation” and a unique and miraculous effect that is superior to any existing detoxification drug proved by the human use experience, can target any single drug and mixed drugs, the advantages and characteristics thereof are that it has universality, short treatment period, quick effect, low cost, detoxification in one week, keeping people strong and healthy in another week, and no withdrawal syndrome, no drug dependence, no toxic side effects and good anti-relapse effect; it is a traditional Chinese medicine compound preparation with pure herb, which has efficacy, safety and clinical benefits; and it can effectively and safely detoxify, strengthen the body resistance and keep fit within 12 days.

The invention and its embodiments are described above, this description is not restrictive, and what is shown in the accompanying drawing is only one of the embodiments of the invention, and the actual structure is not limited to this. All in all, if those skilled in the art receives its enlightenment, without deviating from the object of the invention, and without creatively designing structures and embodiments similar to the technical scheme of the invention shall fall within the protection scope of the invention.

1. A traditional Chinese medicine compound preparation with pure herbs for detoxification and Fukang, wherein: it includes the detoxification preparation, Fukang preparation, as well as dryness-removing and pain-relieving preparation;
the detoxification preparation comprises the following raw materials in parts by weight:
Radix panacis Quinquefolii 50-70 g, Rhizoma gastrodiae 30-35 g, Rhizoma cimicifugae 30-40 g, Radix aconiti lateralis praeparata 5-10 g, pericarpium papaveris 50-70 g, Radix et Rhizoma glycyrrhizae praeparata cum Melle 30-35 g, cortex phellodendri chinensis 40-45 g, Rhizoma coptidis 40-45 g, Radix aucklandiae 25-30 g, Radix astragali 30-40 g, Rhizoma atractylodis Macrocephalae 20-30 g, Radix angelicae sinensis 25-30 g, Radix bupleuri 20-35 g, pericarpium citri reticulatae 25-25 g, Lignum aquilariae resinatum 25-30 g;
the Fukang preparation comprises the following raw materials in parts by weight:
Radix panacis Quinquefolii 50-70 g, Rhizoma cimicifugae 30-40 g, Rhizoma atractylodis Macrocephalae 30-40 g, Radix bupleuri 15-20 g, Cortex eucommiae 30-40 g, roasted Rhizoma gastrodiae 10-20 g, fructus lycii 30-35 g, Radix angelicae sinensis 30-35 g, Lignum aquilariae resinatum 20-30 g, cortex phellodendri chinensis 40-45 g, Radix astragali praeparata cum Melle 30-40 g, Radix et Rhizoma glycyrrhizae praeparata cum Melle 30-35 g, Rhizoma coptidis 40-45 g, fried Semen ziziphi spinosae 25-30 g;
the dryness-removing and pain-relieving preparation comprises the following raw materials in parts by weight:
Rhizoma Corydalis 15-30 g, Radix paeoniae alba 10-15 g, Radix et Rhizoma Notopterygii 15-20 g.

2. A traditional Chinese medicine compound preparation with pure herbs for detoxification and Fukang of claim 1, wherein:
the detoxification preparation comprises the following raw materials in parts by weight:
Radix panacis Quinquefolii 70 g, Rhizoma gastrodiae 35 g, Rhizoma cimicifugae 30 g, Radix aconiti lateralis praeparata 10 g, pericarpium papaveris 50 g, Radix et Rhizoma glycyrrhizae praeparata cum Melle 35 g, cortex phellodendri chinensis 40 g, Rhizoma coptidis 45 g, Radix aucklandiae 30 g, Radix astragali 40 g, Rhizoma atractylodis Macrocephalae 30 g, Radix angelicae sinensis 30 g, Radix bupleuri 20 g, pericarpium citri reticulatae 25 g, Lignum aquilariae resinatum 30 g;
the Fukang preparation comprises the following raw materials in parts by weight:
Radix panacis Quinquefolii 70 g, Rhizoma cimicifugae 40 g, Rhizoma atractylodis Macrocephalae 40 g, Radix bupleuri 15 g, Cortex eucommiae 40 g, roasted Rhizoma gastrodiae 20 g, fructus lycii 35 g, Radix angelicae sinensis 35 g, Lignum Aquilariae Resinatum 30 g, cortex phellodendri chinensis 45 g, Radix astragali praeparata cum Melle 40 g, Radix et Rhizoma glycyrrhizae praeparata cum Melle 35 g, Rhizoma coptidis 40 g, fried Semen ziziphi spinosae 30 g;
the dryness-removing and pain-relieving preparation comprises the following raw materials in parts by weight:
Rhizoma Corydalis 30 g, Radix paeoniae alba 15 g, Radix et Rhizoma Notopterygii 20 g.

3. A preparation method of the traditional Chinese medicine compound preparation with pure herbs for detoxification and Fukang according to claim 1, wherein: its preparation method includes Die Wan manufacturing process and capsule manufacturing process, the detoxification preparation and Fukang preparation adopt the Die Wan manufacturing process, the dryness-removing and pain-relieving preparation adopt the capsule manufacturing process;
The Die Wan manufacturing process comprises the following steps:
Step 1. Inspection: carefully inspecting the formula, drug quality and quantity;
Step 2. Pre-treatment of Pulverization: sorting out the Chinese herbal medicines to be crushed, and putting them in a drying room or oven to dry according to different requirements; drying the medicines at 80° C. for 48 hours, during which the medicines were turned 2-3 times to prevent uneven drying and paste;
Step 3. Pulverization: cutting the dried medicines into the same size and mixing them evenly for pulverization; then putting them into a pulverizer to pulverize and sift; repeatedly pulverizing and sifting until all the medicine powder reaches a fineness of 80-100 mesh;
Step 4. Die Wan: according to the weight of the medicines and the size of the ready-made medicine, preparing a certain amount of medicine, the method is as follow: putting a small amount of medicine powder into the sugar-coated pan, spraying water on the powder with a sprayer, starting the pan, using the rotation of the pan and manual kneading to make the medicine powder evenly moisten, stopping the water and adding powder, so that the medicine powder becomes fine particles, sifting repeatedly in this way to shape the medicinal pills;
then washing and drying the pot, putting it into the medicinal base, dissolving the pot washing water and the remaining materials from the preparation of medicinal base together into a small water paste, for honey pills, adding honey to the honey pill and dissolving it for later use; using slurry or clean water to moisten the medicinal base, then adding powder and rotating continuously to make the medicine powder evenly adhere to the medicinal base; in this way, adding water, adding powder, sifting evenly, and repeating these operations in sequence until the powder is used up, the size of the pills is uniform, and the surface is smooth and appreciable;
Step 5. Drying and Shaping: placing the stacked pills into a drying room or oven for drying, the pills must be dried in an environment at 80° C. for 48 hours, after thorough drying, sifting and shaping them;
Step 6. Moistening and Polishing with water: after the pills are shaped and dried, their surface colors may appear different, therefore, the pills need to be moistened with water, this process involves placing the pills into a pot and adding an appropriate amount of water, allowing all pill surfaces to come into contact with the water, after that, removing the pills from the pot and drying them again in an environment at 80° C. for 12 hours, finally, they can be bottle-packaged.
The capsule manufacturing process comprises the following steps:
Step 1. Inspection: carefully inspecting the formula, drug quality and quantity;
Step 2. Pre-treatment of Pulverization: sorting out the Chinese herbal medicines to be crushed, and putting them in a drying room or oven to dry according to different requirements; drying the medicines at 80° C. for 48 hours, during which the medicines were turned 2-3 times to prevent uneven drying and paste;
Step 3. Pulverization: cutting the dried medicines into the same size and mixing them evenly for pulverization; then putting them into a pulverizer to pulverize and sift; repeatedly pulverizing and sifting until all the medicine powder reaches a fineness of 80-100 mesh;
Step 4. Using the capsule filling machine to inject the medicine powder into the “0” capsule;
Step 5. Packaging them in medicine bottles, sealing and saving.

4. The preparation method of the traditional Chinese medicine compound preparation with pure herbs for detoxification and Fukang of claim 3, wherein: in Step 6 of the Die Wan manufacturing process, the quality of water used for moistening should be distilled water.
